(19) United States
(12) Patent Application Publication
(10) Pub. No.: US 2003/0040125 A1
(43) Pub. Date: Feb. 27, 2003

(54) Methods for performing immunological assays

(75) Inventors: Stephanie F. Bernatchez, Woodbury, MN (US); Barbara C. Swenson, North St. Paul, MN (US); Kurt J. Halverson, Lake Elmo, MN (US); Sanjay L. Patil, Minneapolis, MN (US); Anila Prabhu, Woodbury, MN (US); Jerald K. Rasmussen, Stillwater, MN (US)

Correspondence Address: Christopher D. Gram, Office of Intellectual Property Counsel, 3M Innovative Properties Company, P.O. Box 33427, St. Paul, MN, 55133-3427, US

(73) Assignee: 3M Innovative Properties Company

(21) Appl. No.: 09/934,304

(22) Filed: Aug. 21, 2001

(51) Int. Cl. G01N 33/543

(52) U.S. Cl. 436/518

(57) ABSTRACT

Methods for performing immunological assays are provided. The methods allow performing immunological assays with increased sensitivity, increased specificity, or both. Kits for performing immunological assays having increased sensitivity, increased specificity, or both, are also provided.

### BACKGROUND OF THE INVENTION

Immunological assays may be used to detect or quantitate one or more antigens or antibodies (collectively, “target molecules”) in a sample, e.g., a liquid sample such as blood, urine, saliva, or liquid culture media. It may be desirable to detect a particular target molecule if that target molecule is specifically indicative of a particular condition, e.g., pregnancy, the presence of certain types of cells in a culture, certain types of infections, the presence of certain types of tumors, or other health-related conditions. Many such conditions are associated with the presence of at least one condition-specific target molecule.

The immunological affinity between an antigen and an antibody may be exploited to detect or quantitate a particular target molecule. In many cases this affinity may be very specific, i.e., a particular antibody may bind only to one particular antigen. Thus, immunological affinity may be used as a tool to detect a target molecule that is in a complex mixture including many different types of molecules.

Typically, the molecule that is immunologically complementary to the target molecule is immobilized on some type of solid support. If a particular antigen is to be detected, then an antibody known to bind to the target antigen may be immobilized. If a particular antibody is to be detected, then an antigen known to bind to the target antibody may be immobilized. A sample suspected of containing the target molecule is contacted with the solid support, thereby allowing the target molecule, if present in the sample, to bind to its immunological complementary molecule, thereby forming an antigen-antibody complex immobilized on the solid support. The remainder of the sample is washed away, thereby removing substantially all components of the sample that are not part of an antigen-antibody complex.

Next, a labeled antibody is contacted with the solid support, thereby allowing the labeled antibody to bind to the immobilized antigen-antibody complex. The labeled antibody is selected to bind to the target molecule. However, because the remainder of the sample has been washed away, the specificity of the affinity between the target molecule and the labeled antibody does not have to be as great as the specificity of the affinity exploited to form the antigen-antibody complex. Labeled antibody bound to the antigen-antibody complex provides a signal that may be detected, thereby indicating the presence of the antigen-antibody complex immobilized to the solid support and, therefore, the presence of the target molecule in the original sample. The signal may include any type of signal that may be suitable for the particular assay. Suitable signals include fluorescent signals, radioactive signals, chemiluminescent signals or calorimetric signals, such as may be produced by an enzyme-catalyzed reaction.

Variants of immunological assays include the antibody capture assay and the so-called sandwich assay. Variants of each type of assay may be employed to detect either an antigen or an antibody in a sample. Also, any of the immunological assays described herein generally may use any conventional label in order to produce a detectable signal, such as those listed above.

The antibody capture assay may be used to determine whether a particular target antigen is in a sample. A sample suspected of including the target antigen is applied to a solid support under conditions whereby the target antigen becomes bound to the solid support, where it will be available for binding by a detection antibody. Before the target antigen can be detected, the solid support may be treated with a blocking solution in order to reduce non-specific binding of the detection antibody to the solid support. The blocking solution may include a protein, such as albumin, that is selected to reduce non-specific binding between the solid support, as treated with the blocking protein, and the detection antibody. Thus, the detection antibody is allowed to bind to the target antigen but non-specific binding to the solid support is limited. Consequently, any signal detected should only be the result of target antigen from the sample that has been immobilized to the solid support. A solution containing a detection antibody is allowed to contact the support, thereby allowing the detection antibody to bind to any target antigen bound to the solid support. The detection antibody is selected to specifically bind to the target antigen. Any unbound detection antibody is removed by washing. A labeled antibody is allowed to bind to the detection antibody, thereby forming an antigen-detection antibody-labeled antibody complex. The labeled antibody is selected so that it specifically binds to the detection antibody. Also, the labeled antibody carries a label that allows one to detect the antigen-detection antibody-labeled antibody complex. Detection of the label indicates the presence of the antigen-detection antibody-labeled antibody complex immobilized on the solid support. The complex is possible only if the target antigen was present in the original sample.

The antibody capture assay also may be used to determine whether a particular antibody is present in a sample. An antigen selected to specifically bind to the target antibody (the “capture” antigen) is immobilized on the solid support. The support is blocked as described above and the sample is allowed to contact the solid support bearing the immobilized capture antigen, thereby allowing the target antibody to bind to the capture antigen. Unbound sample is substantially removed and the capture antigen-target antibody complex is detected by allowing a labeled antibody to bind to the target antibody, as described above. Detection of the capture antigen-target antibody complex is possible only if the target antibody is present in the original sample.

If more than one capture antigen is immobilized on the solid support, it is possible to screen a single sample for the presence of more than one target antibody. The various capture antigen-target antibody complexes may be detected by using a plurality of labeled antibodies, each labeled antibody selected to be specific for different target antibody and providing a detectable signal distinguishable from the signal provided by the other labeled antibodies.

The sandwich assay may be used to detect the presence of a target antigen in a sample. A capture antibody is selected so that it specifically binds to the target antigen. The capture antibody is bound to the solid support and the support is blocked. A sample suspected of containing the target antigen is allowed to contact the support, thereby allowing any target antigen in the sample to bind to, i.e., be “captured” by, the capture antibody immobilized to the solid support. If the solution contains a mixture of antigens, only those to which the immobilized antibody will bind will be captured. If an appropriate capture antibody is selected, only the target antigen will be captured and any other antigens in the mixture will remain unbound. Any unbound antigens are removed by washing. A detection antibody is allowed to bind to the captured antigen, thereby forming an immunological sandwich (capture antibody-antigen-detection antibody). Another wash removes any unbound detection antibody. Next, a labeled antibody is allowed to bind to the detection antibody and any unbound labeled antibody is removed by yet another wash. The labeled antibody allows one to detect the presence of the capture antibody-antigen-detection antibody-labeled antibody complex. The complex is possible only if the target antigen is present in the original sample.

If more than one capture antibody is immobilized on the solid support, it is possible to screen a single sample for the presence of more than one target antigen. The various capture antibody-target antigen-detection antibody complexes may be detected by using a plurality of labeled antibodies, each labeled antibody selected to be specific for different detection antibody and providing a detectable signal distinguishable from the signal provided by the other labeled antibodies.

Such immunological assays may generate qualitative or quantitative data about the sample. The presence of a detectable signal, with appropriate controls, can establish the presence of the target molecule in a sample. Quantitative measurement of the amount of target molecule in a sample is also possible. Using a series of samples having known concentrations of the target molecule, one can determine the strength of signal that is detectable for each known concentration. The signal data may be extrapolated to generate a standard curve. Samples with unknown concentrations of the target molecule may be assayed and the detected signal from each unknown sample may be compared to the standard curve. In this way, the concentration of the target molecule in the unknown samples may be determined.

One particular type of immunological assay, an enzyme-linked immunosorbent assay, or ELISA, is particularly useful for detecting or quantifying a target molecule, such as an antigen, that is present in a sample at relatively low concentrations. ELISAs can detect small quantities of a target molecule because an ELISA can be a particularly sensitive assay, as will be described more fully below. Accordingly, ELISAs can be a powerful tool for detecting a target molecule present in a sample at low concentration. When a target molecule, such as a particular antigen, is specifically indicative of a particular medical condition, an ELISA detecting such an antigen can be a especially powerful diagnostic tool for that medical condition.

An ELISA can be a very sensitive immunological assay because the labeled antibody includes an enzyme that catalyzes a reaction involving an enzyme substrate that results in a detectable signal, such as a color change. An ELISA may be performed according to either the antibody capture method or the sandwich assay method described above. Also, an ELISA may be used to detect or quantify either a target antigen or a target antibody. In either method, the labeled antibody includes an enzyme as described above and is allowed to bind to the immobilized immunological complex including the target molecule. A solution containing the enzyme substrate is allowed to contact the support, thereby allowing the enzyme substrate to react with the enzyme linked to the labeled antibody. The resulting signal, e.g., a color change, may be monitored, for example, by measuring the absorbance of light of a particular wavelength by the solution. A single enzyme and, therefore, a single labeled antibody, may catalyze many such reactions and, in effect, generate many thousands of detectable signals. In contrast, each labeled antibody having a fluorescent, chemiluminescent or radioactive label is capable of generating only a single detectable signal. Thus, an ELISA is capable of generating a detectable signal when the target molecule is present in very low concentrations because an ELISA detectable signal is amplified by the reaction catalyzed by the enzyme linked to the labeled antibody.

Assay specificity and sensitivity are qualities by which immunological assays are measured. Sensitivity is a measure of the smallest amount of target molecule that can be detected. Assays having greater sensitivity may be particularly desirable when the available sample of target molecule is small, such as when samples containing the target molecule are costly or difficult to obtain. Specificity of the assay is a measure of the selectivity of the signal generated. Ideally, the detectable signal is a result of the presence of the target molecule, through the specific binding interactions such as those described above, for which the assay is designed and any detectable signal generated by non-specific binding interactions is minimized. However, each antibody binds to an antigen with a particular degree of specificity. The specificity of any immunological assay depends, in part, on the specificity of the affinity between the target molecule and the immunologically complementary molecule being used in the assay. For example, non-specific binding by a detection antibody may provide a false positive assay result, thereby erroneously indicating (through non-specific binding) the presence of a target antigen in a sample having no target antigen. Therefore, a detection antibody that binds to a target antigen with a high degree of specificity and with minimal non-specific binding results in a more specific assay. Assays having a high degree of specificity may be particularly desirable when the occurrence of false positive results are undesirable. In order to minimize non-specific binding of antibodies and, therefore, improve the specificity of an assay, the solid support may be treated with a blocking solution, as described above, in order to reduce non-specific binding of non-target molecules to the substrate.

A continuing need exists for immunological assays with improved sensitivity. Also, a continuing need exists for immunological assays with improved specificity.

### SUMMARY OF THE INVENTION

The present invention provides methods of performing immunological assays having improved specificity, improved sensitivity, or both. The methods include performing an immunological assay using a polymeric substrate as a solid support for the assay in which the polymeric support includes azlactone moieties.

The polymeric substrate including azlactone moieties provides an immunological assay with improved specificity because, surprisingly, quenching of unreacted azlactone moieties substantially reduces non-specific binding of antibodies to the substrate without having to perform a blocking step. Azlactone moieties not involved in affixing target molecules to the substrate may be inactivated by hydrolysis or contacting the substrate with a quencher including, for example, a nucleophile. In either case, quenching the unreacted azlactone moieties substantially reduces non-specific binding of antibodies to the substrate without requiring a blocking step. Reduced non-specific binding of antibodies improves the specificity of the assay. Elimination of the blocking step saves the time associated with the blocking step as well as for preparing the blocking solution and reduces the cost of the assay.

The polymeric substrate may provide an immunological assay with improved sensitivity because the substrate may be shrinkable. Thus, any target molecules affixed to the substrate prior to shrinking are concentrated as the projected surface area of the substrate is reduced during the shrinking step of the method of the present invention. Increasing the concentration of the target molecules is one way to increase the sensitivity of the resulting assay. Also, because the substrate may be shrinkable, less of the target molecule is required to elicit a detectable signal and less of the assay reagents, such as detection antibody, labeled antibody and enzyme substrate, may be required to perform the assay.

Therefore, in accordance with the foregoing, the present invention provides a method for detecting one or more antigens in one or more samples, the method including providing a polymeric substrate including azlactone moieties, the azlactone moieties capable of being quenched, thereby reducing the potential for non-specific binding of a detection antibody to the substrate; affixing one or more samples including one or more antigens to at least a portion of the substrate; substantially removing unaffixed sample; substantially quenching unreacted azlactone moieties, thereby reducing the potential for non-specific binding of a detection antibody to the substrate, allowing a first detection antibody to bind to at least one first antigen, the first detection antibody selected to specifically bind to the at least one first antigen; substantially removing unbound first detection antibody; allowing a first labeled antibody to bind to the first detection antibody, the first labeled antibody including a means for producing a first detectable signal and selected to specifically bind to the first detection antibody; substantially removing unbound first labeled antibody; and detecting the first detectable signal; wherein the step of substantially quenching the unreacted azlactone moieties occurs prior to allowing a first detection antibody to bind to at least one first antigen.

The antigen may be affixed to the substrate by forming covalent bonds with the azlactone moieties or by forming ionic bonds with a substrate coating layer including an ionic surface. The antigen may be affixed directly to the substrate or may be bound to a capture antibody that is affixed to the substrate.

Substantially quenching the azlactone moieties may include hydrolyzing the unreacted azlactone moieties or contacting the unreacted azlactone moieties with a quencher, such as a nucleophile.

Samples or capture antibodies may be affixed to the substrate at discrete locations to form binding sites. The binding sites may be arranged to form an array including any suitable number of binding sites such as, for example, 96, 384, 1536 or more binding sites.

The substrate may include a mask layer, a coating layer including an ionic surface, a shrinkable polymeric material, or any combination thereof.

The means for producing a detectable signal may include an enzyme, a fluorescent label, a calorimetric label, a chemiluminescent label or a radioactive label.

The method of the present invention may be designed to detect or quantitate more than one antigen in a particular sample. The detectable signals that are produced as a result of detecting or quantitating each antigen may be distinguishable or indistinguishable, as desired.

In another aspect, the present invention provides a method for detecting one or more antibodies in a sample, the antibodies having affinity for binding to at least one known antigen, the method including providing a polymeric substrate comprising azlactone moieties, the azlactone moieties capable of being quenched, thereby reducing the potential for non-specific binding of a detection antibody to the substrate; affixing a first known antigen to at least a portion of the substrate, the first known antigen selected to have affinity for a first antibody of interest; substantially removing unaffixed antigen; substantially quenching unreacted azlactone moieties, thereby reducing non-specific binding of antibodies to the substrate; contacting a sample including one or more antibodies with the substrate, thereby allowing any first antibody of interest in the sample to bind to the first known antigen; substantially removing unbound antibodies; allowing a first labeled antibody to bind to the first antibody of interest that is bound to the first antigen, the first labeled antibody comprising a means for producing a first detectable signal and selected to specifically bind to the first antibody; substantially removing unbound first labeled antibody; and detecting the first detectable signal; wherein the step of substantially quenching the unreacted azlactone moieties occurs prior to contacting the sample with the substrate.

The known antigen may be affixed to the substrate by forming covalent bonds with the azlactone moieties or by forming ionic bonds with a substrate coating layer including an ionic surface. The known antigen may be affixed directly to the substrate or may be bound to a capture antibody that is affixed to the substrate.

Substantially quenching the azlactone moieties may include hydrolyzing the unreacted azlactone moieties or contacting the unreacted azlactone moieties with a quencher, such as a nucleophile.

Samples, capture antigens or capture antibodies may be affixed to the substrate at discrete locations to form binding sites. The binding sites may be arranged to form an array including any suitable number of binding sites such as, for example, 96, 384, 1536 or more binding sites.

The substrate may include a mask layer, a coating layer including an ionic surface, a shrinkable polymeric material, or any combination thereof.

The means for producing a detectable signal may include an enzyme, a fluorescent label, a colorimetric label, a chemiluminescent label or a radioactive label.

The method of the present invention may be designed to detect or quantitate more than one antibody in a particular sample. The detectable signals that are produced as a result of detecting or quantitating each antibody may be distinguishable or indistinguishable, as desired.

In another aspect, the present invention also provides a kit for detecting at least one target including a polymeric substrate comprising azlactone moieties, the azlactone moieties being configured to be capable of reducing the potential for non-specific binding of molecules to the substrate, a first detection antibody selected to specifically bind to a first target; and a first labeled antibody comprising a means for generating a first detectable signal and selected to specifically bind to the first detection antibody.

The kit may include a capture antibody affixed to the substrate that is selected to specifically bind to a target antigen. Alternatively, the kit may include a capture antigen affixed to the substrate that is selected to specifically bind to a target antibody. The capture antigen may be affixed directly to the substrates or may be bound to a capture antigen that is affixed directly to the substrate. The capture antibody or capture antigen may be affixed to the substrate in one or more discrete locations to form binding sites. The binding sites may be arranged to form an array. The array so formed may include any suitable number of binding sites, for example, 96, 384, 1536 or more binding sites.

The means for generating a detectable signal may include an enzyme, a fluorescent label, a calorimetric label, a chemiluminescent label or a radioactive label.

The substrate may include a mask layer, a coating layer including an ionic surface, a shrinkable material, or any combination thereof

Various other features and advantages of the present invention should become readily apparent with reference to the following detailed description, examples and claims. In several places throughout the specification, guidance is provided through lists of examples. In each instance, the recited list serves only as a representative group and should not be interpreted as an exclusive list.

### DETAILED DESCRIPTION OF THE INVENTION

The present invention provides methods for performing immunological assays. The methods according to the present invention may be used to improve the sensitivity, specificity, or both of immunological assays. Moreover, these improvements in assay performance may be obtained while decreasing the time and cost required to perform the assays.

For purposes of this invention, the following terms shall have the definitions set forth.

“Affix” shall include any mode of attaching a molecule to a substrate. Such modes shall include, without limitation, covalent and ionic bonding, hydrogen bonding, adherence, such as with an adhesive, and physical entrapment onto or within a substrate. Such modes also shall include attaching a molecule to a substrate indirectly, such as through an intermediate molecule affixed to the substrate. An example of such indirect affixing of a molecule to a substrate includes, but is not limited to, affixing an antigen to a substrate by allowing the antigen to bind to a antibody that is covalently attached to the substrate.

“Antigen” shall mean any chemical molecule, compound, composition or complex to which an antibody has specific affinity. The term “antigen” shall include immunoglobulins, so that, depending upon the function of an immunoglobulin in a particular assay, a single immunoglobulin may be considered to be an antibody, an antigen, or both.

“Binding site” shall mean a discrete location on a substrate at which one or more molecules are affixed. A single binding site may include any quantity of one or more different molecules. For example, a binding site may include a plurality of identical or similar capture antibodies affixed to the substrate. Alternatively, a binding site may include a sample of unknown composition having a plurality of different molecules included therein.

“Molecule” shall include any chemical molecule, compound, composition or complex employed in performing an assay according to the present invention, whether such molecule acts a functional extension of the substrate or is, or is a portion of, a sample of unknown composition. As nonlimiting examples, at least each of the following are considered to be molecules herein: all antigens including target antigens, capture antigens, and non-target antigens in samples of known or unknown composition; all antibodies including capture antibodies, target antibodies, non-target antibodies in samples of known or unknown composition, detection antibodies, and labeled antibodies; or any combination of any of the foregoing.

“Projected surface area” shall mean the area of a surface as calculated with respect to the plane encompassing the “x” and “y” axes of the surface.

“Quenching” shall mean substantially inactivating unreacted azlactone moieties with regard to subsequent chemical reaction, whether covalent, ionic or affinity-based. Quenching may occur by hydrolyzing unreacted azlactone moieties or by contacting the unreacted azlactone moieties with a solution including a quencher, e.g., a nucleophile. Quenching may be completed, in some cases, in as little as two minutes, although longer incubations are also possible. In contrast, methods in which a substrate is blocked, such as with a blocking protein, e.g., albumin, require incubations of at least 30 minutes in order to effectively reduce non-specific binding.

“Target” refers to any molecule, as defined herein, or population of molecules that is to be detected or quantified according to the methods of the present invention. A target molecule may include a heterogeneous or homogeneous population of molecules. For example, a single detection antibody may recognize more than one antigen. The heterogeneous population of antigens recognized by the detection antibody may be considered to be a single target.

“Topographical surface area” shall mean the area of a surface as calculated with respect to the “x”, “y” and “z” axes of the surface.

“Unreacted azlactone moieties” shall mean those azlactone moieties that have not covalently reacted with either 1) a molecule, including but not limited to a portion of a sample, any antigen or any antibody, or 2) a portion of a coating layer.

The present invention includes performing immunological assays using a solid support comprising a polymeric substrate that includes azlactone moieties. Suitable polymeric substrates include those reported in International Publication Nos. WO 99/53319, published Oct. 21, 1999, and WO 01/16370, published Mar. 8, 2001, and U.S. Ser. No. 09/519,450, filed Mar. 5, 2000; U.S. Ser. No. 09/708,916, filed Nov. 8, 2000; and U.S. Ser. No. 09/845,946, filed Apr. 30, 2001. Such substrates include azlactone moieties, may be shrinkable, and may possess additional features that may be exploited in the methods of the present invention, as will be described more fully below. However, the polymeric substrate need not be shrinkable or, if shrinkable, need not be shrunk in order to practice the methods of the present invention.

The polymeric substrate including azlactone moieties may be used as a solid support for many known immunological assays, including, but not limited to, the antibody capture assay and the sandwich assay, each described above. For either type of assay, the polymeric substrate allows performance of the immunological assay with improved specificity, improved sensitivity, or both.

The methods of the present invention are useful for detecting target antigens or target antibodies (collectively, “targets”) present in a sample. A sample may be in any suitable form. Liquid samples may be particularly easy to handle and thoroughly contact with the substrate. A sample may have known or unknown composition and may include one or more targets either alone or in a mixture with other molecules including non-target antigens or non-target antibodies. Suitable samples include, but are not limited to, liquids, liquified food samples, cell culture media, cell lysates, and body fluids such as blood, urine, saliva, cerebrospinal fluid, plasma, and the like. A sample may be purified, partially purified, or unpurified.

Antigen Detection

The antibody capture assay may be used to detect the presence or quantitate the amount of a target antigen in a sample. A portion of the sample is applied to a polymeric substrate including azlactone moieties under conditions such that one or more antigens in the sample including, but not limited to, the target antigen become affixed to the substrate. The sample may be affixed through covalent bonds to the azlactone moieties or through ionic bonds to a coating layer including an ionic surface, described more fully below. Regardless of the particular mode of affixing the sample to the substrate, the substrate may include unreacted azlactone moieties after the sample has been affixed. Unaffixed sample is substantially removed from the substrate by any suitable means.

The unreacted azlactone moieties are substantially quenched in order to reduce the potential for non-specific binding of antibodies to the substrate. In some embodiments, the quenching may occur simultaneously with the step of substantially removing unaffixed sample. However, in other embodiments, the unaffixed sample may be substantially removed in a step separate from quenching the unreacted azlactone moieties. Quenching may include hydrolyzing the unreacted aziactone moieties such as by contacting the unreacted azlactone moieties with water. Distilled or deionized water may help reduce signal interference by, for example, salts, surfactants or other additives, particularly when the detectable signal is a fluorescent signal. Alternatively, quenching may include contacting the unreacted azlactone moieties with a solution including one or more quenchers. Suitable quenchers include, but are not limited to, nucleophiles such as ethanolamine, ammonia and glycine. Consequently, quenching reduces the extent to which molecules, e.g., detection antibodies or labeled antibodies, may subsequently bind to the unreacted azlactone moieties and, therefore, the substrate. As a result, the likelihood and extent of non-specific binding of such molecules to the substrate is reduced, thereby improving the specificity of the assay.

Quenching the unreacted azlactone moieties provides the methods of the present invention with an improved step for inactivating the substrate with regard to non-specific binding. Quenching substantially inactivates the unreacted azlactone moieties but does not affect the covalent attachment of an affixed sample or a coating layer, described more fully below, to reacted azlactone moieties. Quenching may be accomplished by contacting the unreacted azlactone moieties with water. Consequently, no time or expense is required to prepare, store, measure, add, remove or dispose of a blocking solution, such as those containing protein, e.g., albumin. Furthermore, quenching the unreacted azlactone moieties according to the method of the present invention does not require a prolonged incubation. Unreacted azlactone moieties may be quenched in as little as two minutes. In contrast, blocking a solid support with a protein solution in order to reduce non-specific binding may require a minimum incubation time of 30 minutes or, in some cases, many hours.

Quenching the unreacted azlactone moieties should more effectively reduce non-specific binding of a detection antibody to the substrate if quenching occurs prior to allowing the detection antibody to contact the substrate. As used herein, “prior to” means at some time before allowing the detection antibody to contact the substrate. There is no requirement in the methods of the present invention that quenching occur immediately prior to allowing the detection antibody to contact the substrate. Also, quenching after affixing molecules to the substrate may reduce the potential for quenching to interfere with affixing molecules covalently to aziactone moieties, when covalent affixing of molecules to the substrate is desired.

A solution including a detection antibody known to bind to the target antigen is allowed to contact the substrate, thereby allowing the detection antibody to bind to any target antigen that is affixed to the substrate. The detection antibody is selected so that it will bind specifically to the target antigen. As used herein, “bind specifically” means having specific affinity for and is considered in the context of the reaction mixture in which the target antigen is presented to the detection antibody. For example, while a particular detection antibody may be capable of binding to an antigen in addition to the target antigen, if that additional antigen is substantially absent while the detection antibody is allowed to bind to the target antigen according to the method of the present invention, the detection antibody may be considered to specifically bind to the target antigen. Alternatively, a detection antibody may bind to two or more related target antigens, but not bind to other, unrelated antigens. Accordingly, an assay according to the methods of the present invention may be designed to exploit this fact. In such an assay, the detection antibody is considered to bind specifically to the two or more related antigens. Also, one skilled in the art will recognize that antibodies considered to have specific affinity for a particular antigen may exhibit non-specific binding under certain conditions. Thus, it is not required that the detection antibody provide absolute, exclusive affinity for the target antigen in order to be considered to specifically bind to the target antigen.

After unbound detection antibody is removed, a labeled antibody is allowed to bind to the detection antibody that is bound to the target antigen. The labeled antibody is selected so that it will specifically bind to the detection antibody. Again, “specifically bind” is considered with respect to the reaction mixture in which the detection antibody is presented to the labeled antibody. For example, detection antibodies may be raised in a particular species, such as a mouse. The labeled antibodies may be raised so that they will bind to abroad range of immunoglobulins raised in the species in which the detection antibody was raised (in this example, a mouse). Thus, labeled antibodies may not be very specific in a general sense. However, in the context of the reaction mixture in which the detection antibody is presented to the labeled antibody in the methods of the present invention, the specificity of the labeled antibody is sufficient to bind specifically to the detection antibody.

The labeled antibody includes a means for producing a detectable signal, described in more detail below. After unbound labeled antibody is removed, the detectable signal is detected and, in some embodiments of the methods of the present invention, quantified. A signal indicates that the labeled antibody bound to detection antibody, thereby indicating that detection antibody bound to target antigen, thus indicating that target antigen was present in the sample and became affixed to the substrate. The amount of target antigen in the sample may be determined by comparing the amount of signal detected from a sample with a standard curve extrapolated from the signal detected from various known amounts of target antigen.

In some embodiments, more than one target antigen may be detected in a single assay. For such assays, a plurality of detection antibodies may be used to detect various target antigens. The plurality of detection antibodies may be contacted with the substrate one at a time or, alternatively, as a mixture of two or more detection antibodies. Each detection antibody, and therefore each target antigen, may be independently identified, quantified, or both by using a different labeled antibody having a different means for producing a detectable signal to label each target antigen-detection antibody complex.

The sandwich assay also may be used to detect or quantify the amount of a target antigen in a sample. A capture antibody selected so that it specifically binds to the target antigen is affixed to the substrate. Unaffixed capture antibody is removed. The capture antibody may be selected so that it binds to the target antigen, but will not bind to other antigens that may be in the sample. Unreacted azlactone moieties may be quenched prior to contacting the sample with the substrate, thereby reducing the potential for target antigen to covalently react with azlactone moieties. Quenching may occur as a separate step or be combined with the step of removing unaffixed capture antibody. Quenching prior to allowing the detection antibody to contact the substrate may reduce the potential for non-specific binding of a detection antibody to the substrate. Also, quenching after the capture antibodies have been affixed to the substrate may reduce the potential for quenching to interfere with covalently affixing capture antibodies to azlactone moieties, when covalent affixing of capture antibodies to the substrate is desired.

The sample is allowed to contact the substrate, thereby allowing any target antigen in the sample to bind to the capture antibody and unbound sample is removed. After unbound sample is removed, detection antibodies that specifically bind to the target antigen are allowed to contact the substrate, thereby allowing the detection antibodies to bind to the target antigen. Subsequent binding of labeled antibody to the detection antibody and detection of the signal produced by the detection antibody are similar to those described above for the antibody capture assay.

In some embodiments, more than one target antigen may be detected in a single assay. For such assays, a variety of capture antibodies may be employed, each designed to bind to a different target antigen. The capture antibodies may be affixed to the substrate at discrete locations, forming binding sites. The binding sites may be arranged on the substrate to form an array, described more fully below. The various target antigens may come from a single sample or multiple samples. In such assays, a plurality of detection antibodies may be used to detect the various target antigens. The plurality of detection antibodies may be contacted with the substrate one at a time or, alternatively, as a mixture of two or more detection antibodies. Each detection antibody, and therefore each target antigen, may be independently identified, quantified, or both by using a different labeled antibody having a different means for producing a detectable signal to label each target antigen-detection antibody complex.

Antibody Detection

A variation of the antibody capture assay described above may be used to detect or quantify the amount of a target antibody in a sample. A capture antigen (an antigen known to bind to the target antibody) is affixed to the substrate. The capture antigen may be selected so that it is known to bind to the target antibody, but will not bind to any other antibodies that may be in the sample. Unaffixed capture antigen is removed and unreacted azlactone moieties are quenched. Quenching may occur as a separate step or be combined with the step of removing unaffixed capture antigen. Quenching prior to allowing the sample to contact the substrate may reduce the potential for non-specific binding of a target antibody to the substrate. Also, quenching after the capture antigen has been affixed to the substrate may reduce the potential for quenching to interfere with covalently affixing the capture antigen to azlactone moieties, when covalent affixing of the capture antigens to the substrate is desired. After unbound capture antigen is removed and the unreacted azlactone moieties are quenched, the sample is allowed to contact the substrate, thereby allowing any target antibody in the sample to bind to the capture antigen. The unbound portion of the sample is removed and a labeled antibody is allowed to specifically bind to the target antibody bound to the capture antigen. A detectable signal from the labeled antibody indicates that the labeled antibody bound to the target antibody. The amount of target antibody in the sample may be determined by comparing the amount of signal detected from a sample with a standard curve extrapolated from the signal detected from various known amounts of target antibody allowed to bind to an excess amount of capture antigen.

In another embodiment of the methods of the present invention, a sample including one or more antibodies including the target antibody may be affixed to the substrate. Unaffixed sample is removed and unreacted azlactone moieties are quenched. Quenching may occur as a separate step or be combined with the step of removing unaffixed sample. A detection antibody is selected that specifically binds to the target antibody. Quenching prior to allowing the detection antibody to contact the substrate may reduce the potential for non-specific binding of the detection antibody to the substrate. Also, quenching after the sample has been affixed to the substrate may reduce the potential for quenching to interfere with covalently affixing the sample to azlactone moieties, when covalent affixing of the sample to the substrate is desired. After unbound sample is removed and the unreacted azlactone moieties are quenched, the detection antibody is allowed to contact the substrate and, therefore, bind to the target antibody. In some embodiments of the methods of the present invention, a detection antigen known to bind to the target antibody may be contacted with the substrate prior to allowing the detection antibody to contact the substrate. In such embodiments, the detection antibody may specifically bind to the target antibody, the detection antigen, or the target antibody-detection antigen complex. Detection of the target antibodies is as described above for the antigen detection assays.

In some embodiments, more than one target antibody may be detected in a single assay. For some assays, a variety of capture antigens may be employed, each intended to capture a different target antibody. The capture antigens may be affixed to the substrate at discrete locations, forming binding sites. The binding sites may be arranged on the substrate to form an array, described more fully below. For other assays, samples may be affixed to the substrate at discrete locations, forming binding sites. A plurality of detection antigens may be employed to help detect the multiple target antibodies, each detection antigen intended to bind to a different target antibody that has been affixed to the substrate. The binding sites also may be arranged on the substrate to form an array. The various target antibodies may come from a single sample or multiple samples. Each target antibody may be independently identified, quantified, or both by using a different labeled antibody having a different means for producing a detectable signal to label each target antigen-detection antibody complex.

The sandwich assay also may be used to detect or quantify the amount of a target antibody in a sample. A capture antibody is affixed to the substrate and unaffixed capture antibody is removed. The capture antibody is selected to specifically bind to a capture antigen that is, in turn, selected to specifically bind to the target antibody. In some embodiments, it may be possible to select a capture antibody that is known to bind to an epitope of the capture antigen that will not interfere with subsequent binding of the target antibody to the capture antigen. Unreacted azlactone moieties are quenched, as described above. Quenching may occur as a separate step or be combined with the step of removing unaffixed capture antibody. Quenching prior to allowing the capture antigen to bind to the capture antibody may reduce the potential for the capture antigen to covalently bind to the azlactone moieties. Quenching prior to allowing the sample to contact the substrate may reduce the potential for non-specific binding of a target antibody to the substrate. Also, quenching after the capture antibodies have been affixed to the substrate may reduce the potential for quenching to interfere with covalently affixing capture antibodies to azlactone moieties, when covalent affixing the capture antibodies to the substrate is desired.

The capture antigen is allowed to bind to the capture antibody and unbound capture antigen is removed. After unbound capture antigen is removed, the sample is allowed to contact the substrate, thereby allowing any target antibody in the sample to bind to the capture antigen. Subsequent binding of labeled antibody to the target antibody and detection of the signal produced by the detection antibody are similar to those described above for the antibody capture assay.

In some embodiments, more than one target antibody may be detected in a single assay. For such assays, a variety of capture antibodies and capture antigens may be employed, each designed to capture a different target antibody. The capture antibodies may be affixed to the substrate at discrete locations, forming binding sites. The binding sites may be arranged on the substrate to form an array, described more fully below. The various target antibodies may come from a single sample or multiple samples. Each target antibody, may be independently identified, quantified, or both by using a different labeled antibody having a different means for producing a detectable signal to label each target antigen-detection antibody complex.

The Substrate

The substrate includes a polymeric material having azlactone moieties disposed on the polymeric material. The substrate also may include additional features that allow the substrate to be designed to be suitable for performing a particular type of immunological assay. Such additional features are described in greater detail below. Suitable polymeric materials for use in the substrate and the manufacture of a polymeric substrate including azlactone moieties are reported in International Publication No. WO 99/53319. This reference also reports methods of affixing molecules to the polymeric substrate through covalent attachment of the molecules to aziactone moieties. Depending upon the particular assay being performed, capture antigens, capture antibodies or a portion of a sample may be covalently linked to the azlactone moieties of the substrate using methods reported therein.

A substrate suitable for use in the method of the present invention and including a mask layer is reported in International Publication No. WO 01/16370. The mask layer may function to reduce background fluorescence when the labeled antibody includes a fluorescent signal. The reduced background provides for a greater signal to background ratio, which improves the accuracy of assay, particularly when quantitative data are desired.

The substrate may be manufactured to include a coating having an ionic surface. Such substrates are reported in U.S. Ser. No. 09/845,946, filed Apr. 30, 2001. A substrate including an ionic surface allows capture antigens, capture antibodies, a portion of a sample, or any other molecule to be affixed to the ionic coating through ionic bonds rather than being affixed to the substrate through covalent bonds to the azlactone moieties. The ionic coating may include cationic or anionic moieties. Suitable cationic moieties include, but are not limited to, polymers and copolymers made from amine-containing monomers such as 2-vinylpyridine, 3-vinylpyridine, 4-vinylpyridine, (3-acrylamidopropyl)trimethylammonium chloride, 2-diethylaminoethyl acrylate, 2-diethylaminoethyl methacrylate, 3-dimethylaminopropyl acrylate, 3-dimethylaminopropyl methacrylate, 2-aminoethyl methacrylate, dimethylaminoethyl acrylate and methacrylate, 2-acryloxyethyltrimethylammonium chloride, diallyldimethylammonium chloride, 2-methacryloxyethyltrimethylammonium chloride, 3-methacryloxy-2-hydroxypropyltrimethylammonium chloride, 3-aminopropylmethacrylamide, dimethylaminoethyl methacrylamide, dimethylaminopropyl acrylamide, and other similarly substituted acrylamides and methacrylamides; 4-vinylbenzyltrimethylammonium chloride, 4-vinyl-1-methylpyridinium bromide, ethylene imine, lysine, allylamine, vinylamine, nylons and chitosan. Suitable materials for providing an anionic polymeric coating include, but are not limited to, polymers and copolymers of unsaturated acids such as acrylic, methacrylic, maleic, fumaric, itaconic, vinylbenzoic, N-acryloylamino, or N-methacryloylamino acids, 2-carboxyethyl acrylate; vinyl phosphoric acid; vinyl phosphonic acid, monoacryloxyethyl phosphate; sulfoethyl methacrylate; sulfopropyl methacrylate; 3-sulfopropyldimethyl-3-methacrylamidopropylammonium inner salt; styrenesulfonic acid; 2-acrylamido-2-methyl-1-propanesulfonic acid (AMPS); sulfonated polysaccharides such as heparin, dermatan sulfate, and dextran sulfate; carboxylated polyvinyl chloride; and carboxylated polysaccharides such as iduronic acid, carboxymethylcellulose or alginic acid.

For embodiments in which the substrate is manufactured to include a coating having an ionic surface, a portion of the coating may form covalent bonds with a portion of the azlactone moieties of the substrate. The process of applying the coating may include contacting the substrate with an aqueous solution. Thus, unreacted azlactone moieties may be quenched at the same time that the coating is applied to the substrate. Quenching may occur by hydrolysis of unreacted azlactone moieties by water used in the process of depositing the coating layer. Alternatively, the solution used to deposit the coating layer on the substrate may contain one or more quenchers that may contact, and therefore quench, unreacted azlactone moieties.

In some embodiments, the polymeric substrate may be relaxable and, therefore, shrinkable. Relaxable oriented films and relaxable elastomeric materials suitable for use in the substrate of the method of the present invention are reported in International Publication No. WO 99/53319. Relaxable materials may be described as having a projected surface area and a topographical surface area. In a non-relaxed state, the projected surface area and the topographical surface area are substantially equal. However, upon relaxation, the projected surface area is decreased such that the topographical surface area is greater than the projected surface area. Oriented films may be relaxed by the application of energy, such as heat. Elastomeric materials may be held in a stretched condition, then relaxed by removing the force holding the elastomeric material in the stretched condition.

Molecules such as antigens or antibodies that are affixed to a relaxable substrate may be concentrated when the substrate is subsequently relaxed. Certain types of detectable signals, such as fluorescent signals, may be detected more easily in connection with target molecules that have been concentrated as a result of being affixed to a substrate that has been relaxed than they would be in connection with the same target molecules that are affixed to a non-relaxable substrate and, therefore, are unconcentrated. Thus, an immunological assay performed using a relaxed substrate may be able to detect a target molecule present in a sample at a lower concentration than may be detectable performing the same assay using a non-relaxable substrate. The concentration of target molecule may be the same for each assay when the sample is affixed to each substrate. However, relaxing the substrate according to the method of one embodiment of the present invention increases the concentration of the target molecule by decreasing the projected surface area of the substrate. The resulting concentration of target molecules may be sufficient to allow the detection of target molecules affixed to the relaxed substrate that would not be detectable if affixed to a non-relaxable substrate. Consequently, performing immunological assays according to the method of one embodiment of the present invention may increase the sensitivity of the immunological assay. That is, performing an immunological assay according to the method of one embodiment of the present invention may allow the detection of target molecules present in a sample at a lower concentration than is detectable by performing the same immunological assay with another, non-relaxable substrate.

Because a relaxed substrate has a smaller projected surface area, the assay reactions may be conducted in a smaller volume, thereby requiring smaller amounts of reagents such as detection antibodies, labeled antibodies, and, for ELISA assays, enzyme substrates. Also, capture antigens, capture antibodies or samples may be affixed to a relaxable substrate while the substrate is in a non-relaxed form. Thus, molecules may be affixed to the substrate over a relatively large, non-relaxed projected surface area, and still provide the increased assay sensitivity associated with performing the assay over a relatively small, relaxed projected surface area. The ability to affix molecules to the substrate over the larger, non-relaxed projected surface area allows affixing the molecules in more intricately arranged arrays, if desired, while maintaining each area to which molecules are affixed as a discrete binding site, as will be described in more detail below.

Arrays

Molecules may be affixed to the substrate in any suitable pattern or, alternatively, without any discernable pattern. Molecules may be affixed to the substrate in a discrete locations, thereby forming binding sites. A substrate may include any number of binding sites and, when multiple binding sites are present, they may form an array. Certain embodiments may have 96 binding sites on a single substrate. In alternative embodiments, a substrate may include 384 binding sites, 1536 binding sites, or more. The maximum number of binding sites is limited only by the ability to deposit and affix molecules to the substrate while maintaining discrete binding sites. A substrate having a large number of binding sites provides for subjecting a large number of samples to the same assay under identical assay conditions for high throughput analyses.

It may be desirable to include an alignment register site within an array, thereby providing a means by which detected signals may be placed in register with binding sites for proper identification of each binding site and, therefore, the molecules affixed at each binding site providing a detectable signal. Also, alignment registers may allow one to compare the results from arrays on separate substrates. For example, samples may be affixed to two or more substrates in the same pattern, thereby forming a plurality of identical arrays. Each array may be subjected to a different assay, i.e., probed with a different detection antibody. Alignment registers allow the detectable signals from corresponding binding sites on each array to be compared in order to identify the assortment of antigens detectable in each sample.

Each binding site may include any quantity of one or more different molecules. For example, a binding site may include a plurality of identical antibodies affixed to the substrate. Alternatively, a binding site may include a sample having a plurality of different molecules included therein. In another alternative, a binding site may be designed to include a preselected assortment of antibodies (or, alternatively, antigens). One skilled in the art may design a particular binding site in any manner to accomplish the desired immunological detection or quantitation assay.

An array may include identically or similarly designed binding sites. Alternatively, an array may include a plurality of unique binding sites. An array may instead include a plurality of sets of binding sites with the binding sites within each set being identical or similar to the others within the set, but the binding sites in one set being dissimilar to the binding sites in another set. When an array includes binding sites having different molecules affixed to the substrate, a single detection antibody may bind to antigen at one binding site, but not bind to any antigens present at another binding site. As a result, a signal may be detected at some binding sites within an array but not at other binding sites. Such varied possible arrangements provide the ability to perform multiple comparative assays on a single substrate.

It may be possible to design an immunoassay so that a detection antibody binds to more than one antigen. For example, it may be desirable to be able to detect or measure the total amount of two or more closely related antigens in a sample or a collection of samples. Such an assay may be designed to have each antigen detected by a different detection antibody, then computing the sum of the antigen bound by each detection antibody. Alternatively, the labeled antibodies used to detect the target antigen-detection antibody complex may carry detectable signals that are indistinguishable from each other, so that only one signal, representing the sum of the target antigens, is detectable. Indistinguishable signals may still provide differential detection of multiple target molecules however, because, for example, the multiple target antigens may be captured by known capture antibodies located at known positions on the substrate. Therefore, the “address” of each capture antibody, and thus also the target antigen captured by the capture antibody, in an array may be known. A detectable signal at a given location on the array, with appropriate controls, identifies the target antigen that is captured at that location as being present in the sample. In another alternative embodiment, a single detection antibody may detect all of the antigens of interest and the binding of the single detection antibody may be measured as previously described.

It also may be possible to design an immunoassay in which more than one detection antibody is used to probe a single array, thereby permitting detection or quantitation of more than one antigen. Each detection antibody may bind to a different antigen and, in turn, each labeled antibody may bind to a different detection antibody. The labeled antibodies may be selected so that each has a label that produces a detectable signal that is distinguishable from the detectable signals produced by other labeled antibodies. Thus, a plurality of signals may be detected from a single array, thereby indicating the presence of more than one antigen of interest in the samples forming the array. Also, it may be possible to identify certain samples to which more than one detection antibody is bound, indicating that the sample includes more than one antigen of interest. This greatly increases the complexity of immunological assays possible according to the methods of the present invention.

Although characterized in terms of assays for detecting or quantifying one or more target antigens, the assays described above may be similarly modified to provide the ability to detect or quantify one or more target antibodies.

The label on each labeled antibody may be of any suitable type. As used herein, “label” refers to any means for producing a detectable signal, regardless of whether the signal requires one or more additional steps to become detectable. For example, a fluorescent label may require exposure to light of a certain wavelength in order to produce a detectable signal. In another embodiment, an enzyme label may require the presence of an enzyme substrate in order to produce a detectable color change. Suitable labels include, but are not limited to, labels that are fluorescent, calorimetric, chemiluminescent, radioactive or enzymatic.

The label selected for use with a particular assay may, in part, affect the selection of materials used in the substrate. For example, it may be desirable to use a substrate having a mask layer to reduce background fluorescence if a fluorescent label is desired. Certain types of mask layers may interfere with detection of certain types of calorimetric signals. Also, certain relaxable substrates may become opaque upon relaxation. The opaque nature of such substrates may make these substrates less desirable for use in connection with certain colorimetric labels.

The present invention also includes a kit for performing an immunoassay as described above. A kit designed for detection of one or more target antigens generally includes a polymeric substrate having azlactone moieties, a detection antibody selected to specifically bind at least one target antigen, and a labeled antibody selected to specifically bind to at least one detection antibody. The azlactone moieties are configured so that they may be capable of reducing the potential for non-specific binding of molecules to the substrate. In one embodiment, the kit may include a substrate in which the azlactone moieties may be capable of being quenched during or after affixing a sample to the substrate. In other embodiments, the kit may include a substrate in which the unreacted azlactone moieties are quenched during manufacturing, such as during or after affixing one or more capture antibodies to the substrate. When a kit includes more than one labeled antibody, each labeled antibody may include a label that produces a detectable signal that is distinguishable from the detectable signal produced by another labeled antibody. The one or more antigens may be affixed to the substrate, thereby forming binding sites. Alternatively, one or more capture antibodies may be affixed to the substrate to form binding sites. Binding sites may be arranged to form an array. Certain kits may include an array having 96 binding sites, while other embodiments may include an array having 384 binding sites, 1536 binding sites, or more. However, any suitable number of binding sites may be present in an array included in a kit of the present invention. The substrate may include a relaxable material, a mask layer, a coating including an ionic surface, or any combination of the foregoing.

A kit designed for the detection of one or more target antibodies generally includes a polymeric substrate including azlactone moieties. In one embodiment, the kit also includes at least one antigen affixed to the substrate that is selected to specifically bind to at least one target antibody, and at least one labeled antibody selected to specifically bind to at least one target antibody. In an alternative embodiment, the kit may include the polymeric substrate including azlactone moieties, at least one detection antibody selected to specifically bind to the target antibody, and at least one labeled antibody selected to specifically bind to at least one detection antibody. The azlactone moieties are configured so that they may be capable of reducing the potential for non-specific binding of molecules to the substrate. In one embodiment, the kit may include a substrate in which the azlactone moieties may be capable of being quenched during or after affixing a sample to the substrate. In other embodiments, the kit may include a substrate in which the unreacted azlactone moieties are quenched during manufacturing, such as during or after affixing one or more capture molecules to the substrate. Capture antigens selected to specifically bind to a target antibody may be affixed directly to the substrate or may be bound to a capture antibody that is affixed to the substrate. When a kit includes more than one labeled antibody, each labeled antibody may include a label that produces a detectable signal that is distinguishable from the detectable signal produced by another labeled antibody. The antigens affixed to the substrate form discrete binding sites, which may be arranged to form an array. Certain kits may include an array having 96 binding sites, while other embodiments may include an array having 384 binding sites, 1536 binding sites, or more. However, any suitable number of binding sites may be present in an array included in a kit of the present invention. The substrate may include a relaxable material, a mask layer, a coating including an ionic surface, or any combination of the foregoing.

### EXAMPLES

The following examples have been selected merely to further illustrate features, advantages, and other details of the invention. It is to be expressly understood, however, that while the examples serve this purpose, the particular ingredients and amounts used as well as other conditions and details are not to be construed in a matter that would unduly limit the scope of this invention.

### Example 1

Protein Spotting and Detection on Azlactone-functionalized Shrink Film

Three 3 cm×4.75 cm pieces of azlactone-functionalized polyethylene shrink film with a titanium layer (prepared as in Example 2 of International Publication No. WO 01/16370, published Mar. 8, 2001) were spotted in triplicate with 2 μl of human myoglobin (obtained from ICN Pharmaceuticals, Aurora, Ohio, Cat # 55840) at the following concentrations: 100 μg/ml, 10 μg/ml, 5 μg/ml, 1 μg/ml, 0.2 μg/ml, and 0.04 μg/ml in a carbonate buffer (50 mM carbonate, pH 9.5). Bovine serum albumin (BSA, Sigma Chemical Company, St. Louis, Mo., Cat # A9085) was spotted as a negative control at 100 μg/ml in the same carbonate buffer. The drops were allowed to dry at room temperature for 30 minutes. The films were not blocked. Instead, the films were washed with water using a spray bottle until all traces of carbonate salt had disappeared, then dried using a Pasteur pipette hooked to compressed air. The films were shrunk with a heat gun for 10 to 30 seconds to a film temperature of 130° C.

The first film (Film 1) was then contacted with 150 μl of goat antiserum against human myoglobin (ICN Pharmaceuticals, Aurora, Ohio, Cat # 55131) at a 1:300 dilution in PBS buffer (10 mM phosphate, 0.15M NaCl, pH 7.0) containing 0.1% BSA for a 2 hour incubation at room temperature. At the end of this incubation period the solution was removed by aspiration and the film was washed with water and dried as previously described. The film was then contacted with a rabbit anti-goat IgG, Cy3-labeled (Sigma Chemical Company, St. Louis, Mo., Cat #C2821) at a 1:200 dilution in PBS containing 0.1% BSA for a 1.5 hour incubation at room temperature. At the end of this incubation the solution was removed by aspiration and the film was washed with water and dried as previously described. The film was scanned using a GenePix 4000A scanner and the scanned image was analyzed using the GenePix software version 3.0 (Axon Instruments, Foster City, Calif.).

Films 2 and 3 were prepared as negative controls. Film 2 was contacted with an irrelevant goat IgG (Sigma Chemical Company, St Louis, Mo., Cat # I9140) at a 1:200 dilution in PBS containing 0.1% BSA for a 2 hour incubation at room temperature. After the incubation, the solution was removed and Film 2 was washed with water and dried as previously described. Film 2 was then contacted with the Cy3-labeled rabbit anti-goat IgG as described for Film 1. Film 2 was washed and scanned as described for Film 1.

Film 3 was contacted with 150 μl of goat antiserum against human myoglobin as described for Film 1. After the incubation, the solution was removed and Film 3 was washed with water and dried as previously described. Film 3 was then contacted with a Cy3-labeled mouse anti-biotin IgG (Sigma Chemical Company, St. Louis, Mo., Cat # C5585), 1:200 dilution in PBS containing 0.1% BSA under conditions as described for Film 1. Film 3 was washed and scanned as described for Film 1.

Film 1 showed fluorescent spots with decreasing fluorescent intensity in the diluted concentrations of myoglobin spotted on the film. The lowest myoglobin concentration detected under these conditions was 1 μg/ml. No background fluorescence was detected on the BSA spots on Film 1, indicating the substantial absence of nonspecific binding of a) the goat antiserum against human myoglobin, and b) the Cy3-labeled mouse anti-biotin IgG. Films 2 and 3 each were negative.

### Example 2

Protein Capture by Antibody and Detection with a Different Antibody on Azlactone-functionalized Shrink Film

Four 3 cm×4.75 cm pieces of azlactone-functionalized polyethylene shrink film with a titanium layer (prepared as in Example 2 of International Publication No. WO 01/16370, published Mar. 8, 2001) were spotted in triplicate with 2 μl of goat antiserum against human myoglobin (ICN Pharmaceuticals, Aurora, Ohio, Cat. #55131) at the following dilutions: 1:10, 1:100, 1:300 in a carbonate buffer (50 mM carbonate, pH 9.5). An irrelevant goat IgG (Sigma, St. Louis, Mo., Cat #I9140) was spotted as a negative control at a 1:100 dilution in the same carbonate buffer. The drops were allowed to dry at room temperature for 30 minutes, The films were not blocked. Instead, the films were washed with water using a spray bottle until all traces of carbonate salt had disappeared, then dried using a Pasteur pipette hooked to compressed air. The films were shrunk with a heat gun for 10 and 30 seconds to a film temperature of 130° C.

The first film (Film 1) was then covered with 150 μl of human myoglobin (ICI Pharmaceuticals, Aurora, Ohio, Cat #55840) at a concentration of 10 μg/ml in PBS containing 0.1% bovine serum albumin (BSA) for a 1.5 hour incubation at room temperature. At the end of this incubation the solution was removed by aspiration, the film was washed with water and dried as previously described. The film was then contacted with a rabbit antiserum against human myoglobin (Sigma, St. Louis, Mo., Cat. #M8648) at a 1:300 dilution in PBS containing 0.1% BSA for a 1.5 hour incubation at room temperature. At the end of this incubation the solution was removed by aspiration, the film was washed with water and dried as previously described. Film 1 was then contacted with a goat anti-rabbit IgG labeled with Alexa Fluor 633 dye (Molecular Probes, Eugene, Oreg., Cat. #A21070) at a concentration of 10 μg/ml in PBS containing 0.1% BSA for a 1.5 hour incubation at room temperature. At the end of this incubation the solution was removed by aspiration, the film was washed with water and dried as previously described. Film 1 was scanned using the Genomic Solutions LS IV Scanner (Genomics Solutions, Ann Arbor, Mich.). The image was analyzed using GENEPIX software version 3.0 (Axon Instruments, Foster City, Calif.).

Films 2, 3 and 4 were used as negative controls. Film 2 was covered with an irrelevant protein (bovine serum albumin 10 μg/ml, Sigma, St. Louis, Mo., Cat. #A9085) in PBS containing 0.1% BSA for a 1.5 hour incubation at room temperature. The second incubation, third incubation, scanning and analysis of Film 2 were as described for Film 1.

Film 3 was contacted with human myoglobin, incubated, washed and dried as described for Film 1. Film 3 was contacted with an irrelevant rabbit IgG (Sigma, St. Louis, Mo., Cat. #I8140), 1:200 dilution in PBS containing 0.1% BSA, for a 1.5 hour incubation at room temperature. Film 3 was washed and dried as previously described. Film 3 was then contacted with a goat anti-rabbit IgG labeled with Alexa Fluor 633 dye as described for Film 1. The film was washed with water and dried as previously described. Film 3 was scanned and analyzed as described for Film 1.

Film 4 was contacted with human myoglobin, incubated, washed and dried as described for Film 1. Film 4 was then contacted with a rabbit antiserum against human myoglobin (Sigma, St. Louis, Mo., Cat. #M8648), washed with water and dried as described for Film 1. Film 4 was then contacted with an irrelevant IgG labeled with a Cy5 dye, which has the same wavelength as Alexa Fluor 633 dye (FLUOROLINK Cy5 labeled goat anti-mouse IgG, Amersham Pharmacia, Piscataway, N.J., Cat. #PA45002) at a concentration of 10 μg/ml in PBS containing 0.1% PSA. The film was washed with water and dried as previously described. Film 4 was scanned and analyzed as described for Film 1.

Film 1 showed fluorescent spots with decreasing fluorescent intensity in the diluted concentrations of goat antiserum against human myoglobin spotted on the film. No background fluorescence was detected on the spots containing goat IgG. Films 2, 3 and 4 were each negative.

### Example 3

Protein Spotting and Detection of Cell Lysate on Azlactone-functionalized Shrink Film

Azlactone-functionalized polyethylene shrink film with a titanium layer (prepared as in example 2 of International Publication No. WO 01/16370, Mar. 8, 2001) was immersed for five minutes in a solution of polyethyleneimine (PEI, Aldrich Chemical Company, Milwaukee, Wis.) diluted to 0.3% (w/w) with isopropanol. The shrink film was removed from the PEI solution, rinsed with isopropanol, and allowed to dry.

B.forsythus (American Type Culture Collection, Manassas, Va., Catalog #43037) was cultured according to ATCC instructions. Bacteria were harvested and the number of bacteria colony forming units/ml (CFU/ml) was determined by measuring the absorbance at 600 nm. 5×10⁶ CFU/ml of bacteria were used to make a bacterial extract. The bacterial extract was made by heat-killing the bacteria at 70° C. for 1 hour. The bacterial extract was frozen at −70° C. overnight. The extract was then freeze-thawed 4 times using liquid nitrogen. After the freeze-thaw cycles the extract was sonicated for a total of 2 minutes over eight cycles of 15 seconds sonication/15 seconds of rest. The extract was suspended in 0.1M NaHCO₃ buffer (pH 9.0) and 2 μl was spotted onto the PEI-coated azlactone-functionalized shrink film. This spotting was done in triplicate. Additionally non-specific protein (Human Ig, Sigma, St. Louis, Mo.) was coated on the film as a control.

The film was incubated at room temperature overnight and then rinsed with water by spraying the film for 15 seconds. The film was heat shrunk and crosslinked using a UV STRATALINKER 2400 (Stratagene, La Jolla, Calif.) set to “autocrosslink”. After crosslinking it was washed with PBS buffer. The film was blocked using PBS and 5% blocking solution (Kirkegaard Perry Laboratories, Gaithersburg, Md.). The film was incubated for 2 hours at 37° C. with the first detection antibody, rabbit anti-B. forsythus serum. After washing, the film was incubated at 37° C. for 2 hours with goat anti-rabbit IgG labeled with fluorescein isothiocyanate (FITC).

The film was washed and scanned on a GENEPIX 4000A microarray scanner (Axon Instruments, Foster City, Calif.). Spots were observed where the protein molecules (B. forsythus cellular extract) had been spotted indicating that the protein molecules survive the heat-shrink step. Additionally the first detection antibody is capable of recognizing the specific epitopes after the protein had undergone the heat shrink test. Finally the results indicate that protein molecules from cellular extracts are not sloughed off of the shrink film during the shrink process.

The complete disclosures of the patents, patent documents and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method for detecting one or more targets in one or more samples, the method comprising:
providing a polymeric substrate comprising azlactone moieties, the azlactone moieties capable of being quenched, thereby reducing the potential for non-specific binding of a detection antibody to the substrate;
affixing one or more samples comprising one or more targets to at least a portion of the substrate;
substantially removing unaffixed sample;
substantially quenching unreacted azlactone moieties, thereby reducing the potential for non-specific binding of a detection antibody to the substrate;
allowing a first detection antibody to bind to at least one first target, the first detection antibody selected to specifically bind to the at least one first target;
substantially removing unbound first detection antibody;
allowing a first labeled antibody to bind to the first detection antibody, the first labeled antibody comprising a means for producing a first detectable signal and selected to specifically bind to the first detection antibody;
substantially removing unbound first labeled antibody; and
detecting the first detectable signal;
wherein the step of substantially quenching the unreacted azlactone moieties occurs prior to allowing a first detection antibody to bind to at least one first antigen.

2. The method of claim 1 wherein the step of affixing a sample comprising one or more targets to at least a portion of the substrate comprises allowing at least a portion of a sample to form covalent bonds with at least a portion of the azlactone moieties.

3. The method of claim 1 wherein the substrate further comprises a coating layer comprising an ionic surface.

4. The method of claim 3 wherein the coating layer comprises an anionic surface.

5. The method of claim 4 wherein the coating layer comprises at least one polymer made from acrylic acid, methacrylic acid, maleic acid, fumaric acid, itaconic acid, vinylbenzoic acid, N-acryloylamino acid, N-methacryloylamino acid, 2-carboxyethyl acrylate, vinyl phosphoric acid, vinyl phosphonic acid, monoacryloxyethyl phosphate, sulfoethyl methacrylate, sulfopropyl methacrylate, 3-sulfopropyldimethyl-3-methacrylamidopropylammonium inner salt, styrenesulfonic acid, 2-acrylamido-2-methyl1-propanesulfonic acid, carboxylated polyvinylchloride, a sulfonated polysaccharide, a carboxylated polysaccharide, or any combination thereof.

6. The method of claim 3 wherein the coating layer comprises a cationic surface.

7. The method of claim 6 wherein the coating layer comprises at least one polymer made from ethyleneimine, 2-vinylpyridine, 3-vinylpyridine, 4-vinylpyridine, (3-acrylamidopropyl)trimethylammonium chloride, 2-diethylaminoethyl acrylate, 2-diethylaminoethyl methacrylate, 3-dimethylaminopropyl acrylate, 3-dimethylaminopropyl methacrylate, 2-aminoethyl methacrylate, dimethylaminoethyl acrylate, dimethylaminoethyl methacrylate, 2-acryloxyethyltrimethylammonium chloride, diallyldimethylammonium chloride, 2-methacryloxyethyltrimethylammonium chloride, 3-methacryloxy-2-hydroxypropyltrimethylammonium chloride, 3-aminopropylmethacrylamide, dimethylaminoethyl methacrylamide, dimethylaminopropyl acrylamide, 4-vinylbenzyltrimethylammonium chloride, 4-vinyl-1-methylpyridinium bromide, lysine, allylamine, vinylainine, nylons, chitosan, or any combination thereof.

8. The method of claim 3 wherein the step of affixing a sample comprising one or more targets to at least a portion of the substrate comprises allowing at least a portion of the a sample to form ionic bonds with at least a portion of the substrate coating.

9. The method of claim 1 wherein the step of substantially quenching unreacted azlactone moieties comprises hydrolyzing unreacted azlactone moieties or contacting unreacted azlactone moieties with an aqueous solution comprising a quencher.

10. The method of claim 9 wherein the quencher comprises a nucleophile.

11. The method of claim 1 comprising more than one sample wherein each sample is affixed to the substrate at a discrete location.

12. The method of claim 11 wherein the discrete locations form an array.

13. The method of claim 12 wherein the array comprises at least 96 discrete locations.

14. The method of claim 13 wherein the array comprises at least 384 discrete locations.

15. The method of claim 14 wherein the array comprises at least 1536 discrete locations.

16. The method of claim 1 wherein the first target comprises a homogeneous population of molecules.

17. The method of claim 1 wherein the firsttarget comprises a heterogeneous population of molecules.

18. The method of claim 1 wherein the firsttarget is present in two or more samples.

19. The method of claim 1 wherein the substrate comprises a mask layer.

20. The method of claim 1 wherein the polymeric substrate comprises a shrinkable oriented film or a shrinkable elastomeric material.

21. The method of claim 20 further comprising shrinking the polymeric substrate.

22. The method of claim 21 wherein the substrate, after shrinking, comprises a relaxed oriented film or a relaxed elastomeric material.

23. The method of claim 1 wherein the means for producing the first detectable signal is an enzyme, a fluorescent label, a colorimetric label, a chemiluminescent label or a radioactive label.

24. The method of claim 1 wherein the means for producing the first detectable signal comprises an enzyme that is covalently linked to the first labeled antibody.

25. The method of claim 24 wherein the step of detecting the first detectable signal comprises:
allowing an enzyme substrate to react with the enzyme, thereby producing a detectable color change; and
detecting the color change.

26. The method of claim 1 wherein the step of detecting the first detectable signal comprises determining the amount of the first antigen in at least one sample.

27. The method of claim 1 further comprising:
allowing a second detection antibody to bind to at least one second target, the second detection antibody selected to specifically bind to the at least one second target;
substantially removing unbound second detection antibody;
allowing a second labeled antibody to bind to the second detection antibody, the second labeled antibody comprising a means for producing a second detectable signal and selected to specifically bind to the second detection antibody; and
detecting the second detectable signal.

28. The method of claim 27 wherein the step of detecting the second detectable signal comprises determining the amount of second antigen in at least one sample.

29. The method of claim 27 wherein the first detectable signal and the second detectable signal are distinguishable.

30. The method of claim 27 wherein the first detectable signal and the second detectable signal are indistinguishable.

31. A method for detecting a one or more antigens in a sample, the method comprising:
providing a polymeric substrate comprising aziactone moieties, the azlactone moieties capable of being quenched, thereby reducing the potential for non-specific binding of a detection antibody to the substrate;
affixing one or more first capture antibodies to at least a portion of the substrate;
substantially removing unaffixed first capture antibodies;
substantially quenching unreacted azlactone moieties, thereby reducing non-specific binding of a first detection antibody to the substrate;
contacting the sample with the substrate, thereby allowing one or more antigens in the sample to bind to the one or more first capture antibodies;
substantially removing unbound antigens;
allowing a first detection antibody to bind to a bound first antigen, the first detection antibody selected to specifically bind to the first antigen;
substantially removing unbound first detection antibody;
allowing a first labeled antibody to bind to the first detection antibody, the first labeled antibody comprising a means for producing a first detectable signal and selected to specifically bind to the first detection antibody;
substantially removing unbound first labeled antibody; and
detecting the first detectable signal;
wherein the step of substantially quenching the unreacted azlactone moieties occurs prior to allowing a first detection antibody to bind to the first antigen bound to the first capture antibody.

32. The method of claim 31 wherein the step of affixing one or more first capture antibodies to at least a portion of the substrate comprises allowing the capture antibodies to form covalent bonds with the azlactone moieties.

33. The method of claim 31 wherein the substrate further comprises a coating layer comprising an ionic surface.

34. The method of claim 33 wherein the coating layer comprises an anionic surface.

35. The method of claim 34 wherein the coating layer comprises at least one polymer made from acrylic acid, methacrylic acid, maleic acid, fumaric acid, itaconic acid, vinylbenzoic acid, N-acryloylamino acid, N-methacryloylamino acid, 2-carboxyethyl acrylate, vinyl phosphoric acid, vinyl phosphonic acid, monoacryloxyethyl phosphate, sulfoethyl methacrylate, sulfopropyl methacrylate, 3-sulfopropyldimethyl-3-methacrylamidopropylammonium inner salt, styrenesulfonic acid, 2-acrylamido-2-methyl-1-propanesulfonic acid, carboxylated polyvinylchloride, a sulfonated polysaccharide, a carboxylated polysaccharide, or any combination thereof.

36. The method of claim 33 wherein the coating layer comprises a cationic surface.

37. The method of claim 36 wherein the coating layer comprises at least one polymer made from ethyleneimine, 2-vinylpyridine, 3-vinylpyridine, 4-vinylpyridine, (3-acrylamidopropyl)trimethylammonium chloride, 2-diethylaminoethyl acrylate, 2-diethylaminoethyl methacrylate, 3-dimethylaminopropyl acrylate, 3-dimethylaminopropyl methacrylate, 2-aminoethyl methacrylate, dimethylaminoethyl acrylate, dimethylaminoethyl methacrylate, 2-acryloxyethyltrimethylammonium chloride, diallyldimethylammonium chloride, 2-methacryloxyethyltrimethylammonium chloride, 3-methacryloxy-2-hydroxypropyltrimethylammonium chloride, 3-aminopropylmethacrylamide, dimethylaminoethyl methacrylamide, dimethylaminopropyl acrylamide, 4-vinylbenzyltrimethylammonium chloride, 4-vinyl-1-methylpyridinium bromide, lysine, allylamine, vinylamine, nylons, chitosan, or any combination thereof.

38. The method of claim 33 wherein the step of affixing one or more first capture antibodies to at least a portion of the substrate comprises allowing at least a portion of the first capture antibodies to form ionic bonds with at least a portion of the substrate coating.

39. The method of claim 31 wherein the step of substantially quenching the unreacted azlactone moieties comprises hydrolyzing unreacted azlactone moieties or contacting the unreacted azlactone moieties with an aqueous solution comprising a quencher.

40. The method of claim 39 wherein the quencher comprises a nucleophile.

41. The method of claim 31 wherein the substrate further comprising a plurality of discrete binding sites, each binding site defined by an area of the substrate to which a substantially homogeneous population of capture antibodies is affixed.

42. The method of claim 41 wherein the plurality of discrete binding sites forms an array.

43. The method of claim 42 wherein the array comprises at least 96 binding sites.

44. The method of claim 43 wherein the array comprises at least 384 binding sites.

45. The method of claim 44 wherein the array comprises at least 1536 binding sites.

46. The method of claim 41 wherein the first antigen is present at two or more binding sites.

47. The method of claim 31 wherein the first antigen comprises a homogeneous population of molecules.

48. The method of claim 31 wherein the first antibody comprises a heterogeneous population of molecules.

49. The method of claim 31 wherein the substrate comprises a mask layer.

50. The method of claim 31 wherein the polymeric substrate comprises a shrinkable oriented film or a shrinkable elastomeric material.

51. The method of claim 50 further comprising shrinking the polymeric substrate.

52. The method of claim 51 wherein the substrate, after shrinking, comprises a relaxed oriented film or a relaxed elastomeric material.

53. The method of claim 31 wherein the means for producing the first detectable signal is an enzyme, a fluorescent label, a colorimetric label, a chemiluminescent label or a radioactive label.

54. The method of claim 31 wherein the means for producing the first detectable signal comprises an enzyme that is covalently linked to the first labeled antibody.

55. The method of claim 54 wherein the step of detecting the first detectable signal comprises:
allowing an enzyme substrate to react with the enzyme, thereby producing a detectable color change; and
detecting the color change.

56. The method of claim 31 wherein the step of detecting the first detectable signal comprises determining the amount of first antigen in the sample.

57. The method of claim 31 further comprising:
affixing one or more second capture antibodies to at least a portion of the azlactone moieties;
substantially removing unaffixed second capture antibodies;
allowing a second antigen in the sample to bind to the one or more second capture antibodies;
allowing a second detection antibody to bind to the bound second antigen, the second detection antibody selected to specifically bind to the second antigen;
substantially removing unbound second detection antibody;
allowing a second labeled antibody to bind to the second detection antibody, the second labeled antibody comprising a means for producing a second detectable signal and selected to specifically bind to the second detection antibody;
substantially removing unbound second labeled antibody; and
detecting the second detectable signal.

58. The method of claim 57 wherein the substrate further comprising a plurality of discrete binding sites, each binding site defined by an area of the substrate to which a substantially homogeneous population of capture antibodies is affixed.

59. The method of claim 58 wherein at least one binding site comprises first capture antibodies and at least one binding site comprises second capture antibodies.

60. The method of claim 57 wherein the step of detecting the second detectable signal comprises determining the amount of second antigen in the sample.

61. The method of claim 57 wherein the first detectable signal and the second detectable signal are distinguishable.

62. The method of claim 57 wherein the first detectable signal and the second detectable signal are indistinguishable.

63. A method for detecting one or more antibodies in a sample, the antibodies having affinity for binding to at least one known antigen, the method comprising:
providing a polymeric substrate comprising azlactone moieties, the azlactone moieties capable of being quenched, thereby reducing the potential for non-specific binding of a detection antibody to the substrate;
affixing a first known antigen to at least a portion of the substrate, the first known antigen selected to have affinity for a first antibody of interest;
substantially removing unaffixed antigen;
substantially quenching unreacted azlactone moieties thereby reducing non-specific binding of antibodies to the substrate;
contacting a sample including one or more antibodies with the substrate, thereby allowing any first antibody of interest in the sample to bind to the first known antigen;
substantially removing unbound antibodies;
allowing a first labeled antibody to bind to the first antibody of interest that is bound to the first antigen, the first labeled antibody comprising a means for producing a first detectable signal and selected to specifically bind to the first antibody;
substantially removing unbound first labeled antibody; and
detecting the first detectable signal;
wherein the step of substantially quenching the unreacted azlactone moieties occurs prior to contacting the sample with the substrate.

64. The method of claim 63 wherein the step of affixing the first known antigen to at least a portion of the substrate comprises allowing at least a portion of the first known antigen to form covalent bonds with the azlactone moieties.

65. The method of claim 63 wherein the substrate further comprises a coating comprising an ionic surface.

66. The method of claim 65 wherein the coating layer comprises an anionic surface.

67. The method of claim 66 wherein the coating layer comprises at least one polymer made from acrylic acid, methacrylic acid, maleic acid, fumaric acid, itaconic acid, vinylbenzoic acid, N-acryloylamino acid, N-methacryloylamino acid, 2-carboxyethyl acrylate, vinyl phosphoric acid, vinyl phosphonic acid, monoacryloxyethyl phosphate, sulfoethyl methacrylate, sulfopropyl methacrylate, 3-sulfopropyldimethyl-3-methacrylamidopropylammonium inner salt, styrenesulfonic acid, 2-acrylamido-2-methyl-1-propanesulfonic acid, carboxylated polyvinylchloride, a sulfonated polysaccharide, a carboxylated polysaccharide, or any combination thereof.

68. The method of claim 65 wherein the coating layer comprises a cationic surface.

69. The method of claim 68 wherein the coating layer comprises at least one polymer made from ethyleneimine, 2-vinylpyridine, 3-vinylpyridine, 4-vinylpyridine, (3-acrylamidopropyl)trimethylammonium chloride, 2-diethylaminoethyl acrylate, 2-diethylaminoethyl methacrylate, 3-dimethylaminopropyl acrylate, 3-dimethylaminopropyl methacrylate, 2-aminoethyl methacrylate, dimethylaminoethyl acrylate, dimethylaminoethyl methacrylate, 2-acryloxyethyltrimethylammonium chloride, diallyldimethylammonium chloride, 2-methacryloxyethyltrimethylammonium chloride, 3-methacryloxy-2-hydroxypropyltrimethylammonium chloride, 3-aminopropylmethacrylamide, dimethylaminoethyl methacrylamide, dimethylaminopropyl acrylamide, 4-vinylbenzyltrimethylammonium chloride, 4-vinyl-1-methylpyridinium bromide, lysine, allylamine, vinylamine, nylons, chitosan, or any combination thereof.

70. The method of claim 65 wherein the step of affixing the first known antigen to at least a portion of the substrate comprises allowing at least a portion of the first known antigen to form an ionic bond with at least a portion of the substrate coating.

71. The method of claim 63 wherein the step of affixing the known antigen to at least a portion of the substrate comprises:
affixing a capture antibody to the substrate, the capture antibody selected to specifically bind to the first known antigen;
substantially removing unbound capture antibody; and
allowing the first known antigen to bind to the bound capture antibody.

72. The method of claim 63 wherein the step of substantially quenching the unreacted azlactone moieties comprises hydrolyzing unreacted azlactone moieties or contacting the unreacted azlactone moieties with an aqueous solution comprising a quencher.

73. The method of claim 72 wherein the quencher comprises a nucleophile.

74. The method of claim 63 wherein the substrate further comprising a plurality of discrete binding sites, each binding site defined by an area of the substrate to which a substantially homogeneous population of antigens is affixed.

75. The method of claim 74 wherein the plurality of discrete binding sites forms an array.

76. The method of claim 75 wherein the array comprises at least 96 binding sites.

77. The method of claim 76 wherein the array comprises at least 384 binding sites.

78. The method of claim 77 wherein the array comprises at least 1536 binding sites.

79. The method of claim 63 wherein the first antibody of interest is present at two or more binding sites.

80. The method of claim 63 wherein the substrate comprises a mask layer.

81. The method of claim 63 wherein the polymeric substrate comprises a shrinkable oriented film or a shrinkable elastomeric material.

82. The method of claim 81 further comprising shrinking the polymeric substrate.

83. The method of claim 82 wherein the substrate, after shrinking, comprises a relaxed oriented film or a relaxed elastomeric material.

84. The method of claim 63 wherein the means for producing the first detectable signal is an enzyme, a fluorescent label, a colorimetric label, a chemiluminescent label or a radioactive label.

85. The method of claim 63 wherein the means for producing the first detectable signal comprises an enzyme that is covalently linked to the first labeled antibody.

86. The method of claim 85 wherein the step of detecting the first detectable signal comprises:
allowing an enzyme substrate to react with the enzyme, thereby producing a detectable color change; and
detecting the color change.

87. The method of claim 63 wherein the step of detecting the first detectable signal comprises determining the amount of first antibody of interest in the one sample.

88. The method of claim 63 further comprising:
affixing a second known antigen to at least a portion of the substrate, the second known antigen selected to have affinity for a second antibody;
allowing the second antibody to bind to the second known antigen;
allowing a second labeled antibody to bind to the second antibody bound to the second antigen, the second labeled antibody comprising a means for producing a second detectable signal and selected to specifically bind to the second antibody;
substantially removing unbound second labeled antibody; and
detecting the second detectable signal.

89. The method of claim 88 wherein the step of detecting the second detectable signal comprises determining the amount of the second antibody of interest in the sample.

90. The method of claim 88 wherein the first detectable signal and the second detectable signal are distinguishable.

91. The method of claim 88 wherein the first detectable signal and the second detectable signal are indistinguishable.

92. A kit for detecting at least one target comprising:
a polymeric substrate comprising azlactone moieties, the azlactone moietiesconfigured to be capable of reducing the potential for non-specific binding of a detection antibody to the substrate;
a first detection antibody selected to specifically bind to a first target; and
a first labeled antibody comprising a means for generating a first detectable signal and selected to specifically bind to the first detection antibody.

93. The kit of claim 92 wherein the azlactone moieties are quenched or are capable of being quenched.

94. The kit of claim 92 further comprising a first capture molecule affixed to the substrate and selected to specifically bind to the first target.

95. The kit of claim 94 wherein the first capture molecule comprises an antibody and the first target comprises an antigen.

96. The kit of claim 94 wherein the first capture molecule comprises an antigen and the first target comprises an antibody.

97. The kit of claim 94 wherein the first capture molecule is affixed to the substrate at a plurality of binding sites, the binding sites forming an array.

98. The kit of claim 97 wherein the array comprises at least 96 binding sites.

99. The kit of claim 98 wherein the array comprises at least 384 binding sites.

100. The kit of claim 99 wherein the array comprises at least 1536 binding sites.

101. The kit of claim 92 wherein the means for generating a first detectable signal is an enzyme, a fluorescent label, a colorimetric label, a chemiluminescent label or a radioactive label.

102. The kit of claim 101 further comprising an enzyme substrate that, when allowed to react with the enzyme, produces a first detectable color change.

103. The kit of claim 92 wherein the substrate comprises a mask layer.

104. The kit of claim 92 wherein the substrate comprises a coating comprising an ionic surface.

105. The kit of claim 104 wherein the coating layer comprises an anionic surface.

106. The kit of claim 105 wherein the coating layer comprises at least one polymer made from acrylic acid, methacrylic acid, maleic acid, fumaric acid, itaconic acid, vinylbenzoic acid, N-acryloylamino acid, N-methacryloylamino acid, 2-carboxyethyl acrylate, vinyl phosphoric acid, vinyl phosphonic acid, monoacryloxyethyl phosphate, sulfoethyl methacrylate, sulfopropyl methacrylate, 3-sulfopropyldimethyl-3-methacrylamidopropylammonium inner salt, styrenesulfonic acid, 2-acrylamido-2-methyl-1-propanesulfonic acid, carboxylated polyvinylchlioride, a sulfonated polysaccharide, a carboxylated polysaccharide, or any combination thereof.

107. The kit of claim 104 wherein the coating layer comprises a cationic surface.

108. The kit of claim 107 wherein the coating layer comprises at least one polymer made from polyethyleneimine, 2-vinylpyridine, 3-vinylpyridine, 4-vinylpyridine, (3-acrylamidopropyl)trimethylammonium chloride, 2-diethylaminoethyl acrylate, 2-diethylaminoethyl methacrylate, 3-dimethylaminopropyl acrylate, 3-dimethylaminopropyl methacrylate, 2-aminoethyl methacrylate, dimethylaminoethyl acrylate, dimethylaminoethyl methacrylate, 2-acryloxyethyltrimethylammonium chloride, diallyldimethylammonium chloride, 2-methacryloxyethyltrimethylammonium chloride, 3-methacryloxy-2-hydroxypropyltrimethylammonium chloride, 3-aminopropylmethacrylamide, dimethylaminoethyl methacrylamide, dimethylaminopropyl acrylamide, 4-vinylbenzyltrimethylammonium chloride, 4-vinyl-1-methylpyridinium bromide, lysine, allylamine, vinylamine, nylons, chitosan, or any combination thereof.

109. The kit of claim 92 wherein the polymeric substrate comprises a shrinkable material.

110. The kit of claim 92 further comprising:
a second detection antibody selected to specifically bind to a second target; and
a second labeled antibody comprising a means for generating a second detectable signal and selected to specifically bind to the second detection antibody.

111. The kit of claim 110 further comprising a second capture molecule affixed to the substrate and selected to specifically bind to the second target.

112. The kit of claim 110 wherein the means for generating a second detectable signal is an enzyme, a fluorescent label, a colorimetric label or a radioactive label.

113. The kit of claim 112 further comprising an enzyme substrate that, when allowed to react with the enzyme, produces a second detectable color change.

114. The kit of claim 110 wherein the first detectable signal and the second detectable signal are distinguishable.

115. The kit of claim 110 wherein the first detectable signal and the second detectable signal are indistinguishable.

116. A kit for detecting at least one target antibody comprising:
a polymeric substrate comprising azlactone moieties, the azlactone moietiesconfigured to be capable of reducing the potential for non-specific binding of a detection antibody to the substrate;
at least one first capture antigen affixed to the substrate, the first capture antigen selected to specifically bind to a first target antibody; and
a first labeled antibody comprising a means for generating a first detectable signal and selected to specifically bind to the first target antibody.

117. The kit of claim 116 further comprising:
at least one second capture antigen affixed to the substrate, the second capture antigen selected to specifically bind to a second target antibody; and
a second labeled antibody comprising a means for generating a second detectable signal and selected to specifically bind to the second target antibody.
